(19) United States
(12) Patent Application Publication
(10) Pub. No.: US 2022/0079671 A1
(43) Pub. Date: Mar. 17, 2022

(54) SYSTEM AND METHODS FOR INSERTION DEPTH TRACKING

(71) Applicant: Covidien LP, Mansfield, MA (US)

(72) Inventors: Premkumar Rathinasabapathy Jagamoorthy, Telangana (IN); Prathyusha Salandri, Hyderabad (IN); K V S Manoj Kumar Vadali, Hyderabad (IN)

(21) Appl. No.: 17/460,438

(22) Filed: Aug. 30, 2021

Related U.S. Application Data
(60) Provisional application No. 63/077,770, filed on Sep. 14, 2020.

(51) Int. Cl. A61B 18/18

(52) U.S. Cl. CPC A61B 18/1815; A61B 2018/00738

(57) ABSTRACT

A system and an ablation probe of a system monitors insertion depth during an ablation procedure. The ablation probe includes a housing, an elongated shaft extending from the housing, a distance sensor operably coupled to the housing, and a microcontroller operably coupled to the distance sensor. The elongated shaft is configured to be inserted through a skin surface. The distance sensor measures a distance value between the housing and the skin surface. The microcontroller receives the measured distance value from the distance sensor, calculates an insertion depth value of the elongated shaft based on the received measured distance value, and causes a display unit to display the calculated insertion depth value.

### CROSS-REFERENCE TO RELATED APPLICATIONS

This application claims the benefit of U.S. Provisional Patent Application Ser. No. 63/077,770, filed on Sep. 14, 2020.

### FIELD

This disclosure provides a system and method for monitoring insertion depth of an ablation probe during a surgical ablation procedure.

### BACKGROUND

Thermal ablation is used to coagulate diseased tissues, for example in solid organs such as the liver. To perform a thermal ablation, the user places a needle through a portion of the solid organ into the targeted diseased tissue. After placement, the needle is energized to heat the surrounding tissue to create an ablation zone encompassing the target. Precise placement of the needle with respect to the diseased tissue minimizes ablation of healthy tissue while ablating the entirety of the diseased tissue and a margin of tissue around the diseased tissue. After the ablation is created, the user will remove the needle from the organ, and optionally, heat the insertion tract during withdrawal of the needle.

### SUMMARY

The disclosure provides a system and method for performing microwave ablation surgical treatment. In particular, the disclosure is directed to a system and method for monitoring insertion depth of an ablation probe during placement of the ablation probe to a target in an ablation procedure.

According to an aspect of the disclosure, an ablation probe includes a housing, an elongated shaft extending from the housing, a distance sensor operably coupled to the housing, and a microcontroller operably coupled to the distance sensor. The elongated shaft is configured to be inserted through a skin surface. The distance sensor measures a distance value between the housing and the skin surface. The microcontroller receives the measured distance value from the distance sensor and calculates an insertion depth value of the elongated shaft based on the received measured distance value.

In an aspect, the microcontroller is operably coupled to a display unit and is further configured to cause the display unit to display the calculated insertion depth value.

In an aspect, the ablation probe is a microwave ablation probe including a microwave antenna disposed at a distal portion of the elongated shaft.

The distance sensor includes at least one of a laser, ultrasonic, or IR sensor.

In an aspect, the microcontroller calculates the insertion depth value by subtracting the received measured distance value from a length of the elongated shaft.

The display unit may be operably coupled to the housing or external to the housing.

In an aspect, the microcontroller is configured to receive a planned insertion depth value and compare the planned insertion depth value to the calculated insertion depth value. The microcontroller may be configured to generate at least one of an audible, tactile, or visual alert when the calculated insertion depth value is equal to the planned insertion depth value. Additionally, or alternatively, the microcontroller may be configured to generate at least one of an audible, tactile, or visual alert when the calculated insertion depth value is greater than the planned insertion depth value.

In an aspect, the microcontroller is configured to cause the display unit to display the calculated insertion depth value in a first condition, when the calculated insertion depth value is less than a first threshold distance value, in a second condition, when the calculated insertion depth value is greater than the first threshold distance value but less than a second threshold distance value, in a third condition, when the calculated insertion depth value is equal to a planned insertion depth value, and in a fourth condition, when the calculated insertion depth value is greater than the planned insertion depth value.

In an aspect, the microcontroller is configured to cause the display unit to display a value corresponding to a distance between the calculated insertion depth value and a planned insertion depth value.

In another aspect of the disclosure, a system includes an ablation probe and a computing device operably coupled to the ablation probe. The ablation probe includes a housing, an elongated shaft extending from the housing, a distance sensor operably coupled to the housing, and a microcontroller operably coupled to the distance sensor and a display unit. The elongated shaft is configured to be inserted through a skin surface. The distance sensor measures a distance value between the housing and the skin surface. The microcontroller receives the measured distance value from the distance sensor and calculates an insertion depth value of the elongated shaft based on the received measured distance value. The computing device is operably coupled to the microcontroller of the ablation probe and is configured to receive the calculated insertion depth value. The computing device includes a display unit configured to display the calculated insertion depth value.

In an aspect, the ablation probe is a microwave ablation probe including a microwave antenna disposed at a distal portion of the elongated shaft.

The distance sensor includes at least one of a laser, ultrasonic, or IR sensor.

In an aspect, the microcontroller calculates the insertion depth value by subtracting the received measured distance value from a length of the elongated shaft.

In an aspect, at least one of the computing device or the microcontroller is configured to receive a planned insertion depth value and compare the planned insertion depth value to the calculated insertion depth value.

In an aspect, at least one of the computing device or the microcontroller is configured to generate at least one of an audible, tactile, or visual alert when the calculated insertion depth value is equal to the planned insertion depth value.

In an aspect, at least one of the computing device or the microcontroller is configured to generate at least one of an audible, tactile, or visual alert when the calculated insertion depth value is greater than the planned insertion depth value.

In an aspect, at least one of the computing device or the microcontroller is configured to cause the display unit to display the calculated insertion depth value in a first condition, when the calculated insertion depth value is less than a first threshold distance value, in a second condition, when the calculated insertion depth value is greater than the first threshold distance value but less than a second threshold distance value, in a third condition, when the calculated insertion depth value is equal to a planned insertion depth value, and in a fourth condition, when the calculated insertion depth value is greater than the planned insertion depth value.

In an aspect, at least one of the computing device or the microcontroller causes the display unit to display a value corresponding to a distance between the calculated insertion depth value and a planned insertion depth value.

In another aspect of the disclosure, an ablation probe includes a housing, an elongated shaft extending from the housing, a laser distance sensor operably coupled to the housing, and a microcontroller operably coupled to the laser distance sensor and a display unit. The elongated shaft has a length and is configured to be inserted through a skin surface. The laser distance sensor is configured to measure a distance value between the housing and a skin surface. The microcontroller is configured to receive the measured distance value from the laser distance sensor, calculate an insertion depth value of the elongated shaft by subtracting the received measured distance value from the length of the elongated shaft, and cause the display unit to display the calculated insertion depth value.

Any of the above aspects and aspects of the disclosure may be combined without departing from the scope of the disclosure.

### BRIEF DESCRIPTION OF THE DRAWINGS

Objects and features of the presently disclosed system and method will become apparent to those of ordinary skill in the art when descriptions of various aspects thereof are read with reference to the accompanying drawings, of which:

FIG. 1 is a schematic diagram of a microwave ablation system in accordance with an aspect of the disclosure;

FIG. 2 is a schematic diagram of a computing device which forms part of the microwave ablation system of FIG. 1 in accordance with an aspect of the disclosure;

FIG. 3 is a schematic diagram of an ablation probe which forms part of the microwave ablation system of FIG. 1 in accordance with an aspect of the disclosure;

FIG. 4A is an illustration of the ablation probe prior to being inserted into tissue;

FIG. 4B is an illustration of the ablation probe after being inserted into tissue;

FIG. 5A is an illustration of a display unit of the ablation probe of FIG. 3 displaying a planned insertion depth;

FIG. 5B is an illustration of the display unit of the ablation probe of FIG. 3 displaying a difference between a calculated insertion depth and the planned insertion depth in a first condition after the ablation probe is inserted through tissue when the difference is less than a first threshold;

FIG. 5C is an illustration of the display of the ablation probe of FIG. 3 displaying a difference between a calculated insertion depth and the planned insertion depth in a second condition when the difference is greater than the first threshold but less that a second threshold;

FIG. 5D is an illustration of the display of the ablation probe of FIG. 3 displaying a difference between a calculated insertion depth and the planned insertion depth in a third condition when the difference is zero;

FIG. 5E is an illustration of the display of the ablation probe of FIG. 3 displaying a difference between a calculated insertion depth and the planned insertion depth in a fourth condition when the difference is greater than the planned insertion depth; and

FIG. 6 is an example graphical user interface displayable on a display of the microwave ablation system.

### DETAILED DESCRIPTION

The disclosure provides a system and method for monitoring the insertion depth of an ablation probe during insertion of the ablation probe through a patient's skin.

The system, or individual components of the system, monitors the insertion depth of the ablation probe's elongated shaft before, and/or during, application of ablation energy to the target, for the purpose of confirming accurate placement of the ablation probe and monitoring the insertion depth of the ablation probe as it is inserted through the patient's skin.

Several user interfaces and modulated audible, tactile, and visual alerts are disclosed for assisting the clinician in ensuring that the ablation probe is inserted into the patient at the correct depth and that the ablation probe is not inserted beyond the depth where the target is located. The disclosed ablation probe includes a distance sensor for measuring the distance from the ablation probe's housing to the patient's skin. The measured distance is subtracted from the known length of the elongated shaft by a microcontroller within the ablation probe or a separate computing device to calculate the depth for which the elongated shaft is inserted through the skin. The calculated depth is compared to a predetermined target depth and the user is notified as to the degree of the difference between the two depths. The notifications enable the user to know what the ablation probe's current insertion depth is, how far the ablation probe is from a target, when the ablation probe is approaching the target, when the ablation probe is at the target, and when the ablation probe has been inserted too far.

Although the disclosure will be described in terms of specific illustrative aspects, it will be readily apparent to those skilled in this art that various modifications, rearrangements and substitutions may be made without departing from the spirit of the disclosure. The scope of the disclosure is defined by the claims appended hereto. As used herein, the term “clinician” refers to any medical professional (e.g., doctor, surgeon, nurse, or the like) or other user of the system involved in planning, performing, monitoring and/or supervising a medical procedure involving the use of the systems and methods described herein.

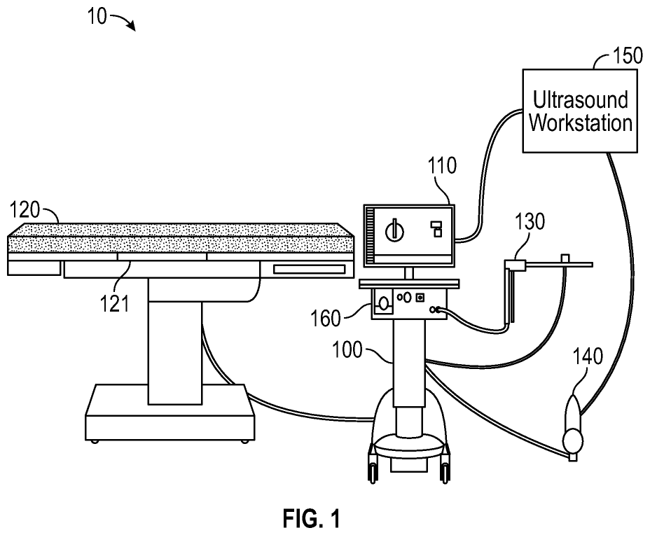

FIG. 1 illustrates a treatment system 10, which includes a computing device 100, a display 110, a table 120, an ablation probe 130, an ultrasound imager 140, an ultrasound workstation 150, and a generator 160. Computing device 100 may be, for example, a laptop computer, desktop computer, tablet computer, or other similar device. Computing device 100 may be configured to control an electrosurgical generator, a peristaltic pump, a power supply, and/or any other accessories and peripheral devices relating to, or forming part of, system 10. Although ultrasound workstation 150 is illustrated in FIG. 1 as a separate component from computing device 100, in aspects, ultrasound workstation 150 may be incorporated into computing device 100 with user interfaces displaying data of ultrasound workstation 150 on a display of computing device 100.

Display 110 is configured to output instructions, images, and messages relating to the performance of the microwave ablation procedure in the form of the graphical user interfaces described below. Although display 110 is shown as a component of computing device 100, in aspects, display 110 may be a separate component of computing device 100, where computing device 100 includes one or more displays for displaying various user interfaces displaying data corresponding to ultrasound data and navigation and ablation parameters and data. Additionally, system 10 may include more than a single display 110. Table 120 may be, for example, an operating table or other table suitable for use during a surgical procedure, which includes an electromagnetic (EM) field generator 121. EM field generator 121 is used to generate an EM field during the microwave ablation procedure and forms part of an EM tracking system which is used to track the positions of surgical instruments, such as ablation probe 130 and ultrasound imager 140, within and around the body of a patient. The EM tracking system (or another tracking system) may additionally include sensors for tracking movement of the patient (e.g., breathing) and may utilize such patient tracking movement to compensate for any displayed elements. Such sensors may include one or more electromagnetic tracking sensors positionable on a patient's chest, which tracks patient body movement independently from any other device (ultrasound wand 140 or ablation probe 130) movements.

Ablation probe 130 is a surgical instrument having a microwave ablation antenna which is used to ablate tissue. In particular, ablation probe 130 may be used to ablate tissue, such as a lesion or tumor (hereinafter referred to as a “target”) by using electromagnetic radiation or microwave energy to heat tissue in order to denature or kill cells, e.g., cancerous cells. The location of ablation probe 130 within the body of the patient may be tracked during the surgical procedure. An example method of tracking the location of ablation probe 130 is by using the EM tracking system, which tracks the location of ablation probe 130 by tracking sensors attached to or incorporated in ablation probe 130.

In addition to the EM tracking system, the surgical instruments may also be visualized by using ultrasound imaging. Ultrasound imager 140, such as an ultrasound wand, may be used to image the patient's body during the microwave ablation procedure to visualize the location of the surgical instruments, such as ablation probe 130, inside the patient's body along with the anatomy of the patient. Ultrasound imager 140 may have an EM tracking sensor embedded within or attached to the ultrasound wand, for example, a clip-on sensor or a sticker sensor. As described further below, ultrasound imager 140 may be positioned in relation to ablation probe 130 such that ablation probe 130 is at an angle to the ultrasound image plane, thereby enabling the clinician to visualize the spatial relationship of ablation probe 130 with the ultrasound image plane and with objects being imaged.

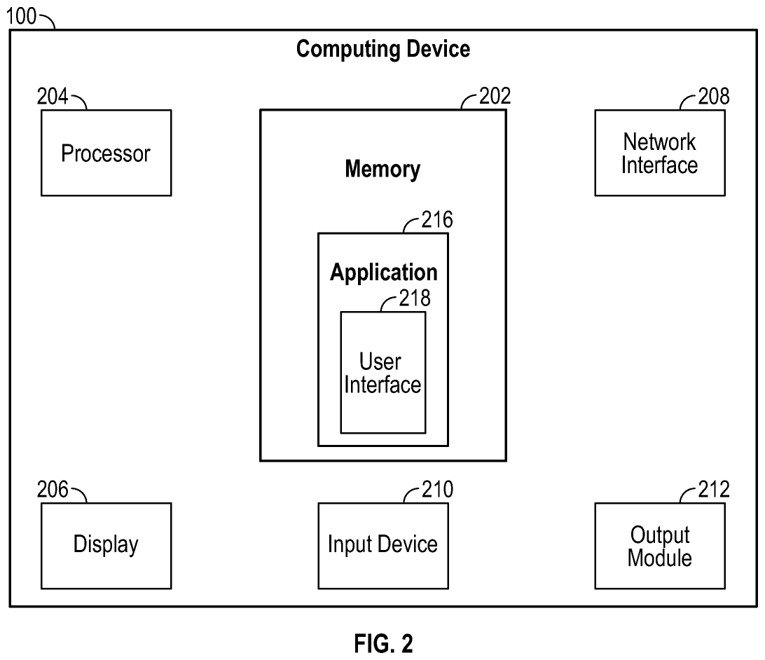

Turning now to FIG. 2, there is shown a system diagram of computing device 100. Computing device 100 may include memory 202, processor 204, display 206, network interface 208, input device 210, and/or output module 212.

Memory 202 includes any non-transitory computer-readable storage media for storing data and/or software that is executable by processor 204 and which controls the operation of computing device 100. In an aspect, memory 202 may include one or more solid-state storage devices such as flash memory chips. Although the description of computer-readable media contained herein refers to a solid-state storage, it should be appreciated by those skilled in the art that computer-readable storage media can be any available media that can be accessed by the processor 204. That is, computer readable storage media includes non-transitory, volatile and non-volatile, removable and non-removable media implemented in any method or technology for storage of information such as computer-readable instructions, data structures, program modules or other data. Memory 202 may store application 216 which may, when executed by processor 204, cause display 206 to present user interface 218 and perform any or all of the steps of the methods described herein.

Processor 204 may be a general purpose processor, a specialized graphics processing unit (GPU) configured to perform specific graphics processing tasks while freeing up the general purpose processor to perform other tasks, and/or any number or combination of such processors.

Display 206 may be touch sensitive and/or voice activated, enabling display 206 to serve as both an input and output device. Alternatively, a keyboard (not shown), mouse (not shown), or other data input devices may be employed.

Network interface 208 may be configured to connect to a network such as a local area network (LAN) consisting of a wired network and/or a wireless network, a wide area network (WAN), a wireless mobile network, a Bluetooth network, and/or the internet. Input device 210 may be any device by means of which a user may interact with computing device 100, such as, for example, a mouse, keyboard, foot pedal, touch screen, and/or voice interface. Output module 212 may include any connectivity port or bus, such as, for example, parallel ports, serial ports, universal serial busses (USB), or any other similar connectivity port known to those skilled in the art.

Application 216 may be one or more software programs stored in memory 202 and executed by processor 204 of computing device 100. As will be described in more detail below, during the planning phase, application 216 guides a clinician through a series of steps to identify a target, size the target, size a treatment zone, and/or determine an access route to the target for later use during the procedure phase. In some aspects, application 216 is loaded on computing devices in an operating room or other facility where surgical procedures are performed, and is used as a plan or map to guide a clinician performing a surgical procedure, but without any feedback from ablation probe 130 used in the procedure to indicate where ablation probe 130 is located in relation to the plan. In other aspects, system 10 provides computing device 100 with data regarding the location of ablation probe 130 within the body of the patient, such as by EM tracking, which application 216 may then use to indicate on the plan where ablation probe 130 are located.

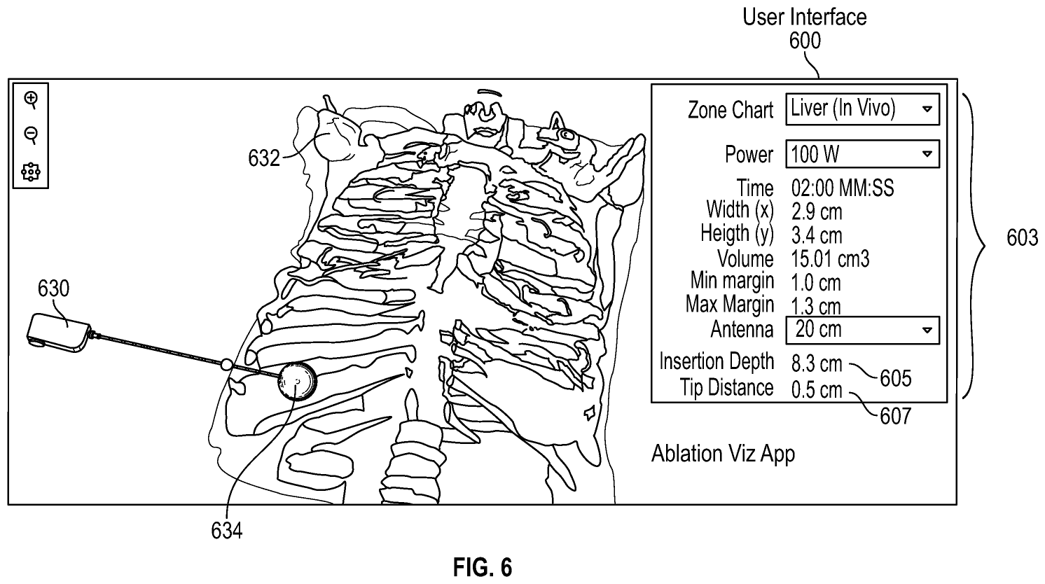

Application 216 communicates with a user interface 218 which generates a user interface for presenting visual interactive features to a clinician, for example, on display 206 and for receiving clinician input, for example, via a user input device. For example, user interface 218 may generate a graphical user interface (GUI) and output the GUI to display 206 for viewing by a clinician. An example of a GUI displayable on display 206 is described below with reference to FIG. 6.

Computing device 100 is linked to display 110, thus enabling computing device 100 to control the output on display 110 along with the output on display 206. Computing device 100 may control display 110 to display output which is the same as or similar to the output displayed on display 206. For example, the output on display 206 may be mirrored on display 110. Alternatively, computing device 100 may control display 110 to display different output from that displayed on display 206. For example, display 110 may be controlled to display guidance images and information during the microwave ablation procedure, while display 206 is controlled to display other output, such as configuration or status information.

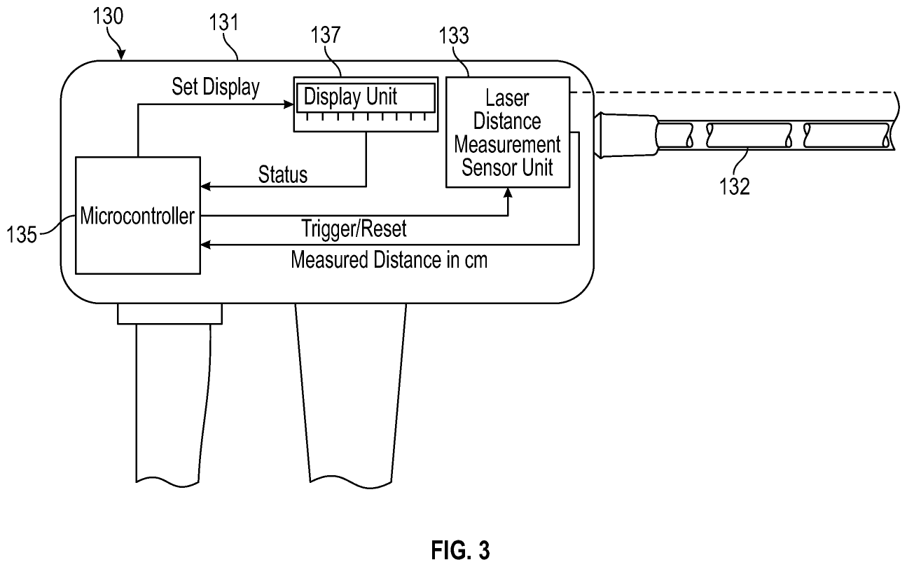

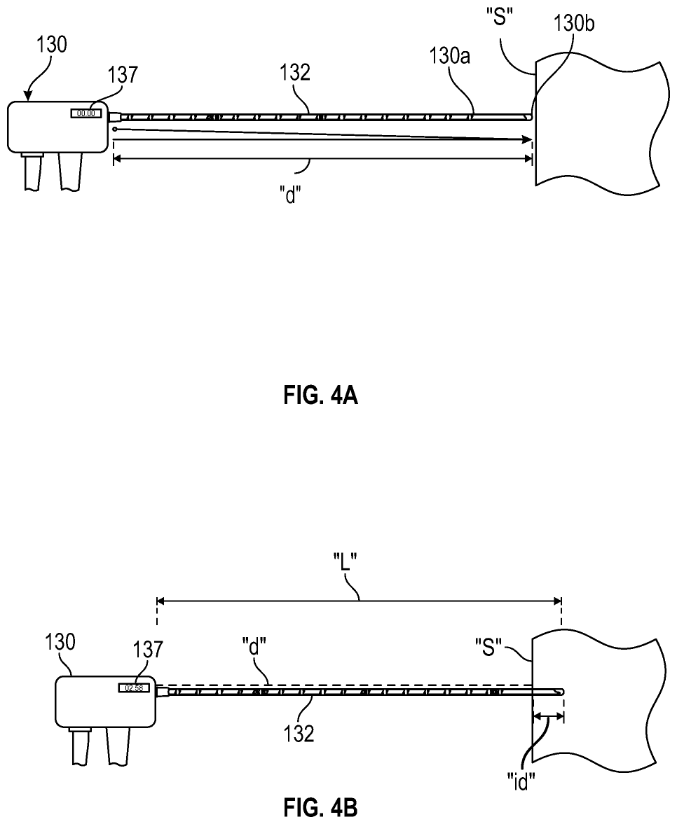

FIG. 3 illustrates the components of ablation probe 130. Ablation probe 130 includes a housing 131 and an elongated shaft 132 extending distally from the housing 131. A microwave antenna 130a (FIG. 4A) is located at a distal portion of the elongated shaft 132 and is configured to emit microwave energy to ablate a target tissue. Additionally, the elongated shaft 132 includes a percutaneous tip 130b (FIG. 4A) for inserting the elongated shaft 132 through a skin surface “S” (FIG. 4A).

Within the housing 131, ablation probe 130 includes a distance sensor 133, a microcontroller 135, and, in some embodiments, a display unit 137. Although microcontroller 135 is illustrated and described as separate from computing device 100 (and processor 204 of computing device 100) (FIG. 2), computing device 100 may function to perform all or some of the functions of microcontroller 135. Similarly, although display unit 137 is illustrated and described as separate from display 110 (FIG. 1), display 110 may function to display all or some of the inicators or user interfaces of display unit 137.

With additional reference to FIGS. 4A and 4B, distance sensor 133 is configured to measure the distance “d” between the housing 131 and skin surface “S.” Distance sensor 133 communicates the measured distance “d” to the microcontroller 135 for further processing by the microcontroller 135. Distance sensor 133 may include at least one of a laser, an ultrasonic, or an infrared (IR) sensor.

When distance sensor 133 utilizes a laser, a laser distance meter emits a pulse of laser at the skin surface “S.” The pulse reflects off the skin surface “S” and back to the distance sensor 133. In this configuration, the distance sensor 133 utilizes a “time of flight” principle, which is based on the fact that laser light travels at a fairly constant speed through the Earth's atmosphere. Utilizing this principle, the distance sensor 133 calculates the distance to skin surface “S.” In particular, the value of the distance “d” between the distance sensor 133 and skin surface “S” is given by d=ct/2, where ‘c’ equals the speed of light and T equals the amount of time for the round trip between the distance sensor 133 and the skin surface “S.” Given the high speed at which the pulse travels and its focus, this rough calculation is very accurate, generally, within an eighth of an inch (3 millimeters) when measuring up to 300 feet (91.5 meters).

The distance sensor 133 may alternatively be an infrared sensor which operates on the principle of reflected light waves to calculate the value of distance “d” to the skin surface “S” or an ultrasonic sensor which operates on the principle of reflected sound waves to calculate the value of distance “d” to the skin surface “S.”

Microcontroller 135 receives the measured distance “d” value from the distance sensor 133 and utilizes that value to calculate the insertion depth “id” of the elongated shaft 132 of the ablation probe 130. In particular, microcontroller 135 subtracts the measured depth value “d” from a known length “L” of the elongated shaft 132. The resulting calculated value (e.g., the difference between the length “L” and the measured depth value “d”) corresponds to the insertion depth “id” value for which the distal portion of the elongated shaft 132 is inserted through the skin surface “S.” Microcontroller 135 compares the calculated insertion depth “id” to one or more threshold values, and/or a predetermined depth corresponding to a predetermined location of a target, to generate audible, visual, or tactile feedback for a user during insertion of elongated shaft 132 through the tissue surface “S” to assist the user in accurately navigating the ablation probe 130 to a target tissue.

Microcontroller 135 can receive a planned insertion depth from a user input or planning software used to identify targets and their locations. Additionally, in some embodiments, microcontroller 135 may be operably coupled to display unit 137 and may cause the display unit 137 to display the value of the calculated insertion depth “id.” The value of the calculated insertion depth “id” may be displayed in different conditions (e.g., different sizes, colors, blinking, steady) based on the degree to which the value of the insertion depth “id” exceeds one or more predetermined thresholds. For example, microcontroller 135 may be configured to cause the display unit 137 to display the calculated insertion depth “id” in a first condition (e.g., a first color), when the calculated insertion depth “id” is less than a first threshold distance value, in a second condition (e.g., a second color) to warn the user that the user is approaching the target when the calculated insertion depth “id” is greater than the first threshold distance value but less than a second threshold distance value, and in a third condition (e.g., a third color) to warn the user that the user has reached the target when the calculated insertion depth “id” is equal to the predetermined insertion depth. Additionally, microcontroller 135 may be configured to cause the display unit 137 to display the calculated insertion depth “id” in a fourth condition (e.g., a fourth color), when the calculated insertion depth “id” is greater than the predetermined insertion depth to notify the user that the user has inserted the ablation probe 130 beyond the target. In addition to displaying the values in different conditions, or as an alternative thereto, microcontroller 135 may generate additional or alternative notifications such as audible, visual, or tactile notifications at any or all of the milestone thresholds described above.

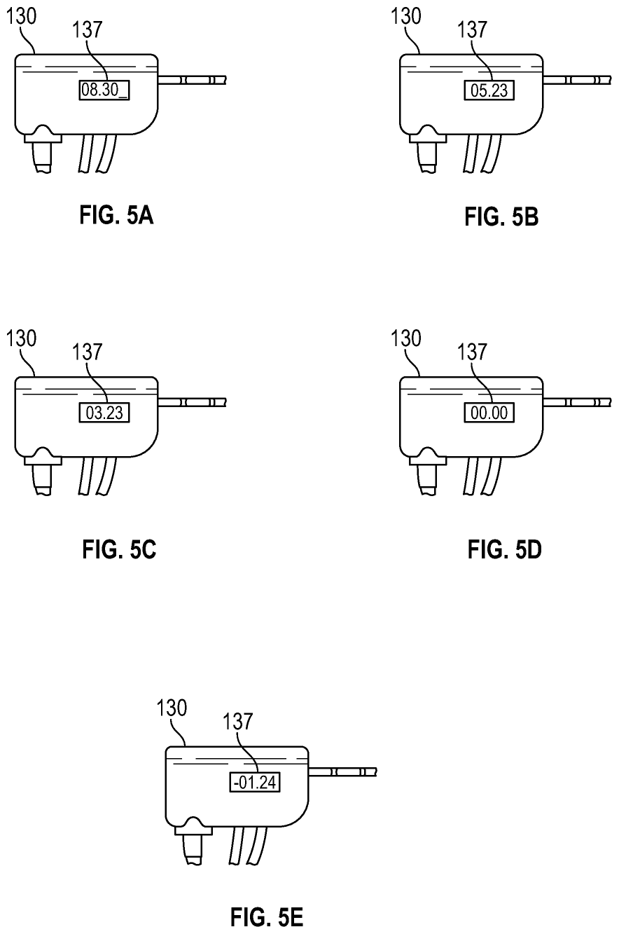

Additionally, microcontroller 135 includes a count-down setting which, after receiving the planned insertion depth, counts-down from the planned insertion depth (e.g., when the distal tip of the elongated shaft is at the skin surface “S”) to zero (e.g., when the distal tip of the elongated shaft 132 arrives at the target) and displays the length remaining to reach the predetermined insertion depth (e.g., the value corresponding to the difference between the calculated insertion depth “id” and the predetermined insertion depth) on display unit 137. The condition of the displayed values on the display unit 137, or any alerts generated by microcontroller 135 may be based on the value corresponding to the length remaining. In particular, FIGS. 5B-5E illustrate examples of conditions in which the display unit 137 may display values to alert a user as to the distance-to-target, which is calculated by subtracting the calculated insertion depth “id” from the predetermined insertion depth. FIG. 5A illustrates the display unit 137 displaying the predetermined insertion depth when the ablation probe 130 is not yet inserted through the skin surface “S.” Such a user interface may be utilized by a user to input the predetermined insertion depth value for processing by the microcontroller 135. FIG. 5B illustrates the display unit 137 displaying the calculated difference after the elongated shaft 132 is inserted through the skin surface “S” in a first condition when the calculated difference is greater than a first predetermined threshold. FIG. 5C illustrates the calculated difference in a second condition when the calculated difference is less than the first predetermined threshold but greater than a second predetermined threshold. FIG. 5D illustrates the calculated difference in a third condition when the calculated difference is zero, indicating that the distal tip of the elongated shaft 132 is at the target (e.g., when the calculated insertion depth “id” is equal to the predetermined insertion depth). FIG. 5E illustrates the display unit 137 displaying the calculated difference in a fourth condition indicating that the user has inserted the ablation probe 130 beyond the target. In addition to displaying the values in different conditions, or as an alternative thereto, microcontroller 135 may generate additional or alternative notifications such as audible, visual, or tactile notifications at any or all of the milestone thresholds described above.

FIG. 6 illustrates an example user interface 600 of computing device 100 which displays parameters 603 of an ablation procedure, the calculated insertion depth “id” value 605, and the remaining distance between the tip of the elongated shaft 132 to a predetermined insertion depth value 607. The user interface 600 also includes a representation of the ablation probe 630 displayed relative to a 3D model of the patient 632 which includes a representation of the target 634. User interface 600 may also be used to calculate or enter an appropriate planned insertion depth to reach the target. During insertion of the ablation probe 130, when the calculated insertion depth “id” is approaching the planned insertion depth, in one aspect, the color of the display changes to alert the user. Additionally, or alternatively, when the tip of the elongated shaft 132 reaches the target location (e.g., when the calculated insertion depth is equal to the planned insertion depth), a portion of user interface 600 (e.g., the “id” value 605) blinks displaying “00 00” cm to alert the user that the tip of the elongated shaft 132 has reached the target. Any further distal advancement to further increase the insertion depth of the ablation probe 130 causes user interface 600 to display a blinking negative value to alert the user that the user has advanced the ablation probe 130 too far. Such a display, or other tactile audible, and/or visual alerts ensures that the user is aware that the ablation probe 130 has been inserted beyond the target or region of interest and that additional caution should be taken (e.g., the user should retract the ablation probe 130 before the ablation probe 130 punctures a critical organ). As described above, any of the functions or displays of user interface 600 may also be displayed on display unit 137 of ablation probe 130 and/or display 110 of system 10.

Although aspects have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing aspects may be made without departing from the scope of the disclosure.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed, entirely or partially, by a single module or unit for purposes of clarity (e.g., computing device 100 and/or microcontroller 135), it should be understood that the techniques of this disclosure may be performed by a combination of units or modules associated with, for example, a medical device, in full or in part (e.g., other components of system 10).

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An ablation probe comprising:
a housing;
an elongated shaft extending from the housing and configured to be inserted through a skin surface;
a distance sensor operably coupled to the housing and configured to measure a distance value between the housing and a skin surface; and
a microcontroller operably coupled to the distance sensor, the microcontroller configured to:
receive the measured distance value from the distance sensor; and
calculate an insertion depth value of the elongated shaft based on the received measured distance value.

2. The ablation probe according to claim 1, wherein the microcontroller is operably coupled to a display unit and is further configured to cause the display unit to display the calculated insertion depth value.

3. The ablation probe according to claim 1, wherein the distance sensor includes at least one of a laser, ultrasonic, or IR sensor.

4. The ablation probe according to claim 1, wherein the microcontroller calculates the insertion depth value by subtracting the received measured distance value from a length of the elongated shaft.

5. The ablation probe according to claim 1, further comprising a display unit operably coupled to the housing.

6. The ablation probe according to claim 1, wherein the microcontroller is configured to receive a planned insertion depth value and compare the planned insertion depth value to the calculated insertion depth value.

7. The ablation probe according to claim 6, wherein the microcontroller is configured to generate at least one of an audible, tactile, or visual alert when the calculated insertion depth value is equal to the planned insertion depth value.

8. The ablation probe according to claim 6, wherein the microcontroller is configured to generate at least one of an audible, tactile, or visual alert when the calculated insertion depth value is greater than the planned insertion depth value.

9. The ablation probe according to claim 1, wherein the microcontroller is configured to cause a display unit to display the calculated insertion depth value:
in a first condition, when the calculated insertion depth value is less than a first threshold distance value;
in a second condition, when the calculated insertion depth value is greater than the first threshold distance value but less than a second threshold distance value;
in a third condition, when the calculated insertion depth value is equal to a planned insertion depth value; and
in a fourth condition, when the calculated insertion depth value is greater than the planned insertion depth value.

10. The ablation probe according to claim 1, wherein the microcontroller is configured to cause a display unit to display a value corresponding to a distance between the calculated insertion depth value and a planned insertion depth value.

11. A system for monitoring insertion depth of an ablation probe, the system comprising:
an ablation probe comprising:
a housing;
an elongated shaft extending from the housing and configured to be inserted through a skin surface;
a distance sensor operably coupled to the housing and configured to measure a distance value between the housing and a skin surface; and
a microcontroller operably coupled to the distance sensor, the microcontroller configured to:
receive the measured distance value from the distance sensor; and
calculate an insertion depth value of the elongated shaft based on the received measured distance value; and
a computing device operably coupled to the microcontroller of the ablation probe and configured to receive the calculated insertion depth value, the computing device including a display unit configured to display the calculated insertion depth value.

12. The system according to claim 11, wherein the ablation probe is a microwave ablation probe including a microwave antenna disposed at a distal portion of the elongated shaft.

13. The system according to claim 11, wherein the distance sensor includes at least one of a laser, ultrasonic, or IR sensor.

14. The system according to claim 11, wherein the microcontroller calculates the insertion depth value by subtracting the received measured distance value from a length of the elongated shaft.

15. The system according to claim 11, wherein at least one of the computing device or the microcontroller is configured to receive a planned insertion depth value and compare the planned insertion depth value to the calculated insertion depth value.

16. The system according to claim 15, wherein at least one of the computing device or the microcontroller is configured to generate at least one of an audible, tactile, or visual alert when the calculated insertion depth value is equal to the planned insertion depth value.

17. The system according to claim 15, wherein at least one of the computing device or the microcontroller is configured to generate at least one of an audible, tactile, or visual alert when the calculated insertion depth value is greater than the planned insertion depth value.

18. The system according to claim 11, wherein at least one of the computing device or the microcontroller is configured to cause the display unit to display the calculated insertion depth value:
in a first condition, when the calculated insertion depth value is less than a first threshold distance value;
in a second condition, when the calculated insertion depth value is greater than the first threshold distance value but less than a second threshold distance value;
in a third condition, when the calculated insertion depth value is equal to a planned insertion depth value; and
in a fourth condition, when the calculated insertion depth value is greater than the planned insertion depth value.

19. The system according to claim 11, wherein at least one of the computing device or the microcontroller causes the display unit to display a value corresponding to a distance between the calculated insertion depth value and a planned insertion depth value.

20. An ablation probe comprising:
a housing;
an elongated shaft extending from the housing and configured to be inserted through a skin surface, the elongated shaft having a length;
a laser distance sensor operably coupled to the housing and configured to measure a distance value between the housing and a skin surface; and
a microcontroller operably coupled to the laser distance sensor and a display unit, the microcontroller configured to:
receive the measured distance value from the laser distance sensor;
calculate an insertion depth value of the elongated shaft by subtracting the received measured distance value from the length of the elongated shaft; and
cause the display unit to display the calculated insertion depth value.
